(19) United States
(12) Patent Application Publication
(10) Pub. No.: US 2013/0071867 A1
(43) Pub. Date: Mar. 21, 2013

(54) Method And Apparatus For Performing Mass Spectrometry

(71) Applicant: Keith Fadgen, Hope Valley, RI (US)

(72) Inventor: Keith Fadgen, Hope Valley, RI (US)

(73) Assignee: WATERS TECHNOLOGIES CORPORATION, Milford, MA (US)

(21) Appl. No.: 13/384,914

(22) PCT Filed: Jul. 16, 2010
(86) PCT No.: PCT/US10/42241

§ 371 Date: Apr. 19, 2012

Related U.S. Application Data
(60) Provisional application No. 61/230,424, filed on Jul. 31, 2009.

(51) Int. Cl. C12M 1/00

(52) U.S. Cl. CPC C12M 45/09
USPC 435/23; 435/287.1

(57) ABSTRACT

The invention feature devices and methods for preserving and processing samples comprising a fluid having deuterated compounds. The device of the present invention comprises a housing defining a first chamber and a second chamber. The first chamber is heated to an elevated temperature and receives the sample and performs a digestion process on the sample at the elevated temperature. The second chamber is cooled to a low temperature and receives the deuterated digested sample from the first chamber and performs one or more separation steps to isolate an analyte. The device of further comprises conduit means for containing and moving the sample into the first chamber to form a digested sample. The conduit means moves the digested sample from the first chamber to the second chamber to separate the sample to form at least one analyte. The analyte is maintained at the low temperature to preserve its deuterated form.

### RELATED APPLICATIONS

This application claims priority to U.S. Provisional Application No. 61/230,424 entitled “Method and Apparatus for Performing Mass Spectrometry” filed on 31 Jul. 2009, the entire contents of which are incorporated herein by reference.

### FIELD OF THE INVENTION

The present invention is directed to a device and a method for preserving and processing one or more samples comprising a fluid having one or more deuterated compounds. And, in particular, embodiments of the present invention feature preserving deuterium-labeled proteins for mass spectral analysis.

### BACKGROUND OF THE INVENTION

The present invention will be described with respect to several terms which are defined below.

As used herein the term ‘sample”, in its broadest sense, refers to compositions of matter for which further information is desired. By way of example, without limitation, the term is used in an analytical, clinical, medical or diagnostic sense to denote one or more groups of compounds, one or more of which may be of interest as to its presence or absence or concentration or form. The compound of interest, often referred to as an analyte, may be compound or compounds for which information is desired, biologically active or inactive, a toxin, biomarker, metabolite, naturally occurring compound, synthetic compound, administered, drug, pro-drug, or drug candidate.

Chromatography is a technique for separating a sample into its constituent parts. It involves passing a sample dissolved in a mobile phase through a stationary phase. A mobile phase can be either a liquid or a gas. Liquid chromatography (LC) uses a liquid as mobile phase. LC can be carried out in a column packed with stationary phase. High performance liquid chromatography (HPLC) and ultra performance liquid chromatography (UPLC) are LC techniques that force a sample solution through a column at a high pressure.

A “detector” is a device or instrument form making measurements of a sample. A mass spectrometer is a detector for ionizing chemical compounds in a sample to generate charged molecules or molecule fragments and determining the mass-to-charge ratios of the charged molecules or molecule fragments. The term LCMS refers to LC coupled with mass spectrometry (MS).

Hydrogen/deuterium exchange mass spectrometry (HXMS) is a special form of mass spectral analysis. In HXMS, hydrogen is exchanged with deuterium and the deuterium acts as a form of label capable of detection by a mass spectrometer. By way of example, without limitation, proteins having hydrogen functional groups which can be exchanged with deuterium can be measured with mass spectrometry. Deuterium is a stable isotope of hydrogen which contains one proton and one neutron. HXMS is used for studying protein conformation.

Hydrogen exists in the deuterium form for a limited period of time before it reverts to the common form. The “quench conditions” or the half-life for deuterium reversion to hydrogen in the 100% H2O environment of HPLC solvent is very quick, on the order of 30-120 min.

As used herein, the term “online” refers to processes which are performed at runtime of an analysis sequence as the sample conveyed from one process to another in a substantially closed flow path of conduits and vessels. This discussion will use the term “conduit means” to refer to such a substantially closed flow path of conduits, pipes, tubes, vessels, columns, fittings, valves, and detectors.

The hydrogen/deuterium exchange reaction is very sensitive to temperature. Higher temperatures produce a faster exchange reaction. The analyte labeled with deuterium becomes unlabeled over time. Therefore, the temperature for all the processing steps must be held to the freezing temperature of deuterated samples so as to slow down the exchange reaction and preserve the deuterium label prior to introducing deuterated samples to a detector.

However, protein and peptide samples are often subjected to digestion processes to produce one or more fragments. These digestion processes are only efficient when performed, typically in digestion columns, at temperatures above freezing. A digestion column is a vessel having an inlet and an outlet with a immobilized enzyme. The enzyme is immobilized by covalent bonding to a solid support held in the vessel. It is preferred to perform digestion processes online in digestion columns maintained above 10 degrees Centigrade and meanwhile to preserve samples in a deuterated form at the freezing point of the samples

### SUMMARY OF THE INVENTION

Embodiments of the present invention are directed to a device and a method for preserving and processing the deuterated forms of proteins and the protein digests for analysis.

One embodiment of the present invention is directed to a device for processing a sample. As used herein, the term “sample” refers to one or more samples comprising a fluid having one or more deuterated proteins. The device of the present invention comprises a housing having one or more walls defining at least a first chamber and a second chamber. The first chamber is heated to an elevated temperature. The first chamber receives the sample and performs a digestion process on the sample at the elevated temperature. The second chamber is cooled to a low temperature. The second chamber receives the sample from the first chamber and performs one or more separation steps to isolate an analyte for further processing at the low temperature. The device of the present invention further comprises conduit means for containing and moving the sample into the first chamber to form a digested sample having one or more deuterated digestion products. The conduit means moves the digested sample from the first chamber to the second chamber to separate the deuterated digestion products to form at least one analyte. The analyte is maintained at the low temperature to preserve its deuterated form.

The terms “elevated temperature” and “low temperature” are relative to each other.

The term “housing” is used in the broadest sense to refer to a single unitary structure. For example, without limitation, such a structure would comprise a cabinet or box with at least one wall forming an enclosure. The structure may preferably comprise one or more platforms or mounting boards for mounting components. In the science of chromatography such a structure is preferably a component known as a sample manager that is placed in a HPLC or an UPLC system or in other analytical instrument configurations. A preferred sample manager manages the injections of one or more samples from plates, vials, syringes, and/or other sampling devices and introduces the samples to a continuously flowing mobile phase that carries the sample into LC columns.

The term “chamber” denotes an interior part of the housing chamber. The chamber is defined by one or more interior walls which form at least a first chamber and a second chamber. The one or more walls forming the first and second chambers may comprise solid, rigid structures or semi solid non-rigid structures. For example, without limitation, the wall may comprise a sheet of metal or glass or composite materials, rigid plastic, flexible plastic or composite sheeting, fabric, thermal fiber insulation, and the like. Preferably, the one or more walls forming the first chamber and second chamber have good thermal insulating properties.

As used herein, the term “one or more separation steps” refers to chromatographic separation steps such as, by way of example, without limitation, trapping or loading of analytes or analyte fragments in one or more columns and further analytical separation on an analytical column. These processes typically shunt undesired materials to waste and desired sample, potentially comprising one or more analytes, to one or more detectors, such as a mass spectrometer. A preferred conduit means includes, but is not limited to, a digestion column, a trapping column, a chromatographic separation column, one or more valves including an injection valve and a switching valve.

In one aspect of the invention, the conduit means further comprises a section of tubing sheathed in a cooled sleeve having a proximal end towards the cooled chamber and a distal end towards an external detector.

Upon completion of the separation of the digested sample, the conduit means conveys at least one analyte from the second chamber to a detector through the section of tubing sheathed in the cooled sleeve to preserve the analyte which may contain one or more the deuterated digestion products. The cooled sleeve is cooled with a dry gas, preferably dry nitrogen, so as to minimize condensation inside of the second chamber. The conduit means further comprises a coupling assembly for placing the distal end of the tubing in fluid communication with the detector.

In one aspect of the invention, a detector is a mass spectrometer. It is contemplated within the scope of present invention that other detection methods such as infrared spectroscopy (IR), Raman spectroscopy, and nuclear magnetic resonance (NMR) may also be used.

In one aspect of the invention, an preferred elevated temperature of the first chamber is selected for the lowest temperature in which the enzyme of the digestion column will exhibit reasonable activity, normally about 10 degrees Centigrade. In one aspect, the low temperature of the second chamber is set to the freezing temperature of the sample. The temperature in the first chamber may be lower than the freezing point of the sample due to co-solvents, pressure effects and salt. The temperature may range of −5 to +5 degrees Centigrade of the freezing temperature.

In one aspect of the invention features a first chamber of the present invention heated by one or more heating devices comprising Peltier units and electrical resistance heaters.

In another aspect, the second chamber is cooled by one or more cooling devices comprising Peltier units, a thermal mass, heat sinks, a resistance temperature detector, insulation cells, and a liquid cooled system.

One aspect of the device of the invention comprises sensing means for sensing the temperature of the first and second chambers. The sensing means produces one or more temperature signals in response to a change in temperature. As used herein, the term “sensing means” refers to electronic and mechanical temperature sensing devices, thermometers, thermostats and the like.

Another aspect of the device of the invention further comprises control means. The control means is in signal communication with the sensing means and the heating and/or cooling devices built in the first and second chambers, and in response to the temperature signals. As used herein the term “control means” refers to computer type controls in the nature of computer processing units (CPUs), personal computing devices, servers, mainframe computers and the like known in the art. As used herein, the term “signal communication” refers to wired, as in electrical signals, or wireless, as in electromagnetic, radio, optical, or infrared transmission devices. The control means issues command signal to the heating and/or cooling devices.

Further embodiments of the present invention are directed to a method of making the device and using the device as described.

### BRIEF DESCRIPTION OF THE DRAWINGS

FIG. 1 is a schematic representation of an embodiment of the apparatus made in accordance with the present invention.

FIG. 2 shows the base peak intensity (BPI) chromatogram of the UPLC separation of peptides resulted from online digestion of deuterated cytochrome c at 0° C. using the device set forth in FIG. 1.

### DETAILED DESCRIPTION OF THE INVENTION

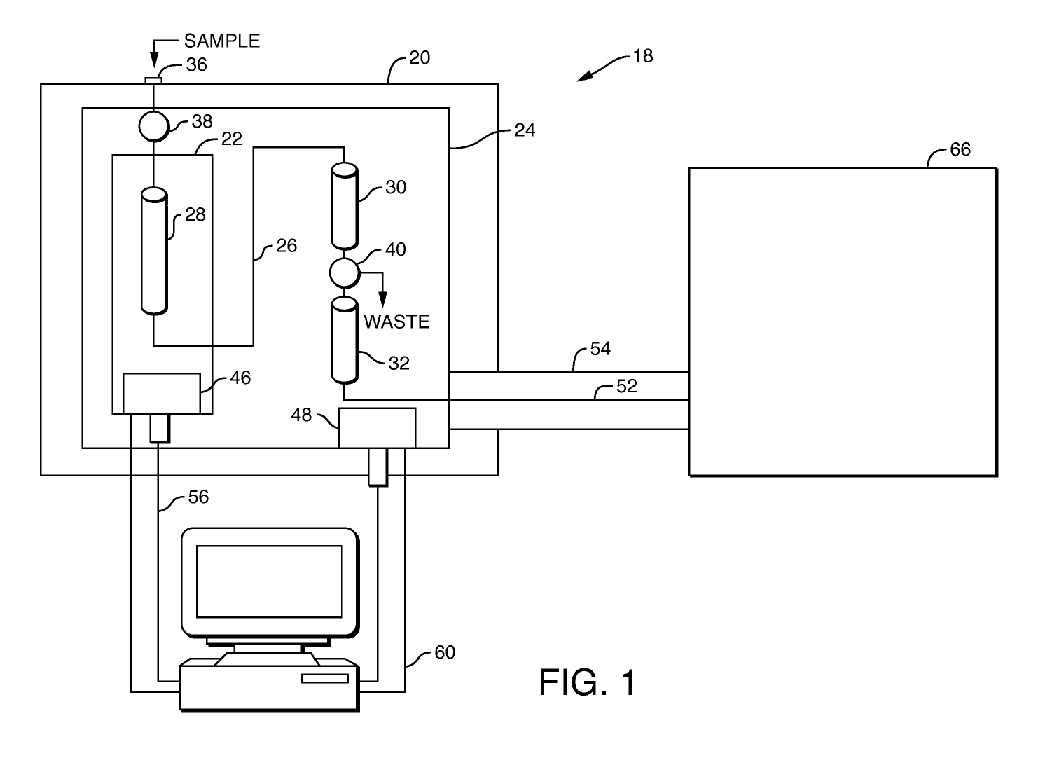

The present invention will now be described with respect to FIG. 1, with the understanding that FIG. 1 and its description are directed to the preferred embodiments of the present invention. Those skilled in the art will recognize that such preferred embodiments are capable of modification and alteration without departing from the teaching of the present disclosure.

Turning now to FIG. 1, a device embodying features of the present invention, generally designated by the numeral 18 in schematic representation, is shown. The device 18 has the following major elements: a housing 20, conduit means 26, control means 60 and sensing means 56. The housing 20 has a first chamber 22 and a second chamber 24.

The first chamber 22 is for receiving one or more samples and performing a digestion process on the sample at an elevated temperature, and the second chamber 24 for receiving the sample from the first chamber and performing one or more separation steps to isolate an analyte for further processing at a low temperature.

The conduit means 26 is for containing and moving the sample having one or more deuterated compounds into the first chamber 22 to form a digested sample containing one or more deuterated digestion products and moving the digested sample from the first chamber 22 to the second chamber 24 to separate the deuterated digestion products to form at least one analyte if present, and the deuterated digestion products and analyte are both maintained at the low temperature.

The control means 60 is for regulating the temperature of the first and second chambers 22 and 24, which is in signal communication with the sensing means 56 and the heating and/or cooling devices built in the first and second chambers 22 and 24, and, in response to one or more temperature signals, issues command signal to the heating and/or cooling devices.

The first chamber 22 is heated to an elevated temperature preferably above 10 degrees Centigrade by one or more heating devices 46 built in the first chamber 22 comprising Peltier units and electrical resistance heaters.

The second chamber 24 is cooled to a low temperature preferably within five degrees of the freezing temperature of the sample by one or more cooling devices 48 built in the second chamber 24 comprising Peltier units, a thermal mass, heat sinks, a resistance temperature detector, insulation cells, and a liquid cooled system.

The temperature of the first and second chambers 22 and 24 is monitored and regulated, by way of example without limitation, by sensing means 56 and control means 60. The sensing means 56 senses the temperature of the first and second chambers 22 and 24 and produces one or more temperature signals in response to a change in temperature. The control means 60 is in signal communication with the sensing means 56 and with the heating and cooling devices 46 and 48 built in the first and second chambers 22 and 24. The control means 60 responses to the temperature signals received from the sensing means 56 and issues command signals to the heating and/or cooling devices 46 and 48.

The conduit means 26 comprises a digestion column 28, a trapping column 30, a chromatographic separation column 32, an injection valve 38 having an injection port 36 exposed on the top of the housing 20, a switching valve 40, and a section of tubing 52 sheathed in a cooled sleeve 54 having a proximal end towards the second chamber 24 and a distal end towards a detector 66. The digestion column 28 is contained in the first chamber 22 maintained at the elevated temperature. The trapping column 30, chromatographic separation column 32, injection valve 38, and switching valve 40 are contained in the second chamber 24 maintained at the low temperature.

The sample comprising a fluid having one or more deuterated proteins is injected through the injection port 36 exposed on the top of the housing 20 and delivered through the injection valve 38 to a continuously flowing mobile phase that carries the sample into the digestion column 28 in the first chamber 22. The sample is digested in the digestion column 28 at the elevated temperature to form a digested sample containing one or more deuterated digestion products. The digested sample is moved by the conduit means 26 from the first chamber 22 to the second chamber 24 to be loaded in the trapping column 30. Undesired materials in the sample are directed to waste from the trapping column 30 through the switching valve 40 and desired sample, potentially comprising one or more analytes, is directed through the switching valve 40 to the chromatographic separation column 32 for separation of individual compounds in the digested sample. The processes of loading and separation are carried out at the low temperature to preserve the deuterated digestion products.

Upon completion of the separation of the digested sample, at least one analyte is formed, which may contain one or more deuterated digestion products. The analyte is conveyed through the conduit means 26 from the second chamber 24 to the section of tubing 52 sheathed in a cooled sleeve 54 to the detector 66. The cooled sleeve 54 is cooled by a dry gas, preferably dry nitrogen, so as to minimize condensation inside of the second chamber 24 and limit the amount of deuterium loss in the tubing 52 connected to the detector 66. The detector 66 is preferably a mass spectrometer.

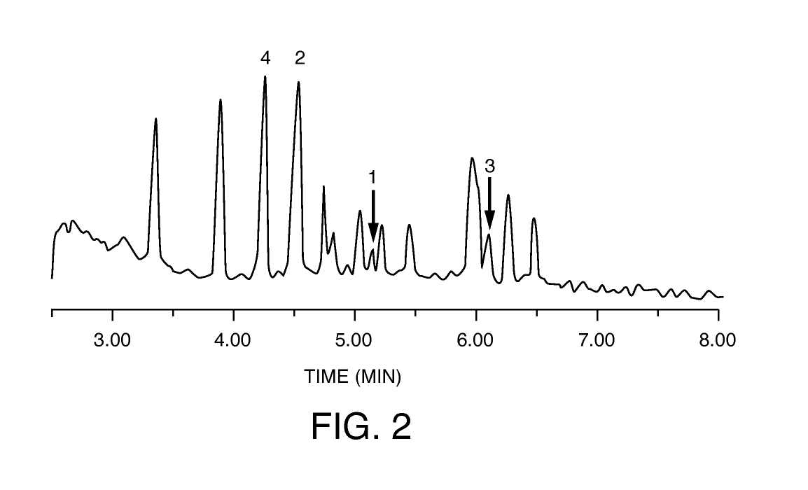

The device and method of the present invention are further described with respect to the following example shown in FIG. 2.

### EXAMPLE

FIG. 2 shows the BPI chromatogram of the UPLC separation of cytochrome c peptides obtained on the device set forth in FIG. 1. The chromatographic peaks denoted by 1, 2, 3 and 4 on the chromatogram shown in FIG. 2 represent four representative peptides from four distinct parts of cytochrome c. 1, peptide 1-10 GDVEKGKKIF; 2, peptide 37-46 GRKTGQAPGF; 3, peptide 67-82 YLENPKKYIPGTKMIF; and 4, peptide 95-104 IAYLKKATNE. The peptides were resulted from online digestion of deuterated cytochrome c, and all the peptides were contained within a 3.5-min elution window at 0° C. The experimental results show that the device of the invention performs better separations of the peptides than a HPLC system while provides the same deuterium incorporation information as obtained with a traditional ice bath-cooled HPLC system.

While this invention has been particularly shown and described with references to specific embodiments, it will be understood by those skilled in the art that various changes in form and details may be made therein without departing from the spirit and scope of the invention as defined by the appended claims.

What is claimed is:

1. A device for processing one or more samples comprising a fluid having one or more deuterated compounds comprising:
a. a housing having one or more walls defining at least a first chamber and a second chamber, said first chamber for receiving said one or more samples and performing a digestion process on said sample at an elevated temperature, said second chamber for receiving said sample from said first chamber and performing one or more separation steps to isolate an analyte for further processing at a low temperature;
b. conduit means for containing and moving said sample having one or more deuterated compounds into said first chamber to form a digested sample having one or more deuterated digestion products and moving said digested sample from said first chamber to said second chamber to separate said one or more deuterated digestion products to form at least one analyte if present, and said deuterated digestion products and analyte being maintained at said low temperature to preserve said deuterated digestion products and analyte.

2. The device of claim 1 wherein said conduit means conveys at least one of said deuterated digestion products and analyte from said second chamber to a detector in a cooled sleeve to maintain said conduit means at said low temperature to preserve said deuterated digestion products and analyte.

3. The device of claim 2 wherein said detector is a mass spectrometer.

4. The device of claim 2 wherein said conduit means has a section of tubing in said cooled sleeve said tubing having a proximal end towards said second chamber and a distal end towards said detector for placing said deuterated digestion products and analyte in said detector.

5. The device of claim 4 further comprising a coupling assembly for placing said distal end of said tubing of conduit means in fluid communication with said detector.

6. The device of claim 1 wherein said conduit means comprises at least one digestion column, said digestion column is in fluid communication with conduit means in said first chamber.

7. The device of claims 1 wherein said conduit means comprises one or more valves including an injection valve having an injection port exposed on the top of said housing.

8. The device of claim 1 wherein said elevated temperature is above 10 degrees Centigrade.

9. The device of claim 1 wherein said low temperature is selected to within five degrees of the freezing temperature of said sample.

10. The device of claim 1 wherein said low temperature is in the range of −5 to 5 degrees Centigrade.

11. The device of claim 1 wherein said second chamber is cooled by one or more cooling devices comprising Peltier units, a thermal mass, heat sinks, a resistance temperature detector, insulation cells, and a liquid cooled system.

12. The device of claim 1 wherein said first chamber is heated by one or more heating devices comprising Peltier units and electrical resistance heaters.

13. The device of claim 1 wherein said cooled sleeve is cooled with a dried gas to minimize condensation inside of said second chamber and limit the amount of deuterium loss in said tubing connected to said detector.

14. The device of claim 1 wherein said conduit means conveys at least one of said deuterated digestion products and analyte from said second chamber to a detector in cooled sleeve to maintain said conduit means at said low temperature to preserve said deuterated compounds and analyte and said nitrogen atmosphere is circulated in said sleeve to cool said conduit means.

15. The device of claim 1 wherein said second chamber has at least one separation column for chromatographically separating at least one digestion product.

16. The device of claim 1 wherein said second chamber has at least one trapping column to trap and remove undesired materials from the digested sample.

17. The device of claim 1 further comprising sensing means for sensing the temperature of at least one of said first chamber and said second chamber and producing one or more temperature signals in response to a change in temperature.

18. The device of claim 1 further comprising control means for regulating the temperature of said first and second chambers, said control means in signal communication with said sensing means and said heating and/or cooling devices built in said first and second chambers, and in response to said one or more temperature signals, said control means issuing command signal to said heating and/or cooling devices.

19. A method of processing one or more samples comprising a fluid having one or more deuterated compounds comprising the steps of:
a. providing a device having a housing and conduit means;
1. said housing having one or more walls defining at least a first chamber and a second chamber, said first chamber for receiving said one or more samples and performing a digestion process on said sample at an elevated temperature, said second chamber for receiving said sample from said first chamber and performing one or more separations steps to isolate an analyte for further processing at a low temperature;
2. said conduit means for containing and moving said sample having one or more deuterated compounds into said first chamber to form a digested sample having one of more deuterated digestion products and moving said digested sample having one of more deuterated digestion products from said first chamber to said second chamber to separate said one or more deuterated digestion products to form at least one analyte, said deuterated digestion products and analyte maintained at said low temperature to preserve said one or more deuterated digestion products and analyte;
b. operating said device to receive a sample having one or more deuterated compounds, make a digested sample having one or more deuterated digestion products, to separate one or more deuterated digestion producta and form at least one analyte for analysis.
